(19) United States
(12) Patent Application Publication
(10) Pub. No.: US 2008/0097281 A1
(43) Pub. Date: Apr. 24, 2008

(54) Ultrasonic Drug-Delivery System

(75) Inventors: Leonid Zusman, Haifa (IL); Michael Goldman, Ramat-Gan (IL); Allan C. Entis, Tel-Aviv (IL)

Correspondence Address: Martin Moynihan;PRTSI, INC., P.O. Box 16446, Arlington, VA, 22215, US

(73) Assignee: Sonanco, LTD, Hefer (IL)

(21) Appl. No.: 10/596,411

(22) PCT Filed: Dec. 15, 2004
(86) PCT No.: PCT/IL04/01133

§ 371 Date: Aug. 27, 2007

Related U.S. Application Data
(60) Provisional application No. 60/529,096, filed on Dec. 15, 2003.

(51) Int. Cl. A61M 37/00; A61M 25/00

(52) U.S. Cl. 604/22; 604/528

(57) ABSTRACT

Apparatus for delivering a drug to a target site of a body comprising: a dispersing member adapted to vibrate when acoustically excited; a source of acoustic energy controllable to couple acoustic energy to the dispersing member to excite it to vibrate; and a drug adhered to the dispersing member so that when the acoustic source excites the dispersing member, the drug is dispersed therefrom.

### RELATED APPLICATIONS

The present application claims the benefit under 35 USC 119(e) of US provisional application 60/529,096 filed on Dec. 15, 2003, the disclosure of which is incorporated herein by reference.

### FIELD OF THE INVENTION

The present invention relates to administration of a substance to a region of the body such as administration of a therapeutic material to a localized region of an organ of the body.

### BACKGROUND OF THE INVENTION

Often in the treatment of disease it is advantageous to deliver a concentrated dose of a therapeutic drug to a localized region of tissue while minimizing contact of the drug with tissue for which the drug is not intended. For example, in treating a malignant brain tumor in a patient, it is generally advantageous to deliver a concentrated anticancer agent to the site of the tumor with minimal contamination of non-cancerous brain tissue with the drug. However, it is generally difficult to achieve such localized administration of a drug to a target site in the brain.

If a drug is administered systemically through the blood stream, penetration of the drug to the brain may be severely hindered by the blood brain barrier. Furthermore, it is generally very difficult to localize quantities of the drug that manage to penetrate the blood brain barrier to the desired site. In addition, many anticancer drugs are hydrophobic. To enable a hydrophobic anticancer drug to dissolve in the blood and be systemically distributed so that it can reach a cancer site, the drug is usually embedded or encapsulated in a hydrophilic material. Some hydrophilic materials, such as for example Cremophor, that are used for embedding and encapsulating drugs are toxic and the toxicity of a material used to embed or encapsulate an anticancer drug, rather than side effects of the drug itself, often limits dosage of the anticancer drug.

In some procedures, to overcome the blood brain barrier a drug is delivered to a target site in the brain by direct invasive application of the drug to the site. For example, in some procedures a transcranial catheter is inserted through a hole drilled in the skull and positioned so that a liquid containing the drug that is transported through the catheter perfuses through brain tissue at the site. However, perfusing a drug into the brain usually requires a relatively long application period during which the transcranial catheter must remain in position in the brain. It is also generally difficult to control spatial distribution of the liquid comprising the drug, as a result of which it is often difficult to limit delivery of the drug that it carries to the desired localized site. Furthermore, whereas a range to which the drug is dispersed is in general limited to less than a few mm from a location at which the liquid is introduced into the brain, it is often desired to deliver a drug to a site characterized by an extent of tens of mm.

In some procedures an anti-tumor drug may also be invasively administered using a “Gliadel wafer”. The Gliadel wafer is formed from a polymer material impregnated with the drug. Typically, a plurality of Gliadel wafers is inserted into a cavity that remains at a site of a tumor after the tumor is removed. After insertion, the polymer material in the wafers slowly dissolves releasing the drug to the affected area over an extended period of time. Dispersion of the drug from the locations of the wafers is limited and a Gliadel wafer is generally not suitable for dispersion of a drug to regions of tissue not contiguous with or close to the wafer.

The problem of administering a medication locally to a desired target region is of course not limited to cancerous sites in the brain. For example, to prevent restenosis it is often desirable to deliver and maintain, for a relatively extended period of time, a concentration of a restenosis inhibiting agent to a localized region of a blood vessel wall. Presently, antirestenosis drugs may be incorporated on a stent inserted into a blood vessel, however, it appears that only limited quantities of such drugs may be effectively incorporated in a stent and stents coated with these drugs are generally expensive.

### SUMMARY OF THE INVENTION

An aspect of some embodiments of the present invention relates to providing a method of delivering a therapeutic substance, hereinafter a “drug”, to a localized target region of an organ, such as for example the brain or a thrombosis site of a blood vessel in a patient.

An aspect of some embodiments of the invention, relates to providing a method of delivering a drug in solid form to a localized target region. Optionally, to provide a solid form of the drug, the drug may, for example, be encapsulated, embedded in, or bonded to the surface of, or an appropriate coating on the surface of, suitable particles. In some embodiments of the invention, the particles are particles characterized by sizes less than a few microns. In some embodiments of the invention, the particles are characterized by sizes less about 250 nanometers. In some embodiments of the invention, the particles are characterized by sizes less than 50 nanometers.

According to an aspect of some embodiments of the invention, the drug and/or particles comprising the drug is adhered to an ultrasonic vibrator, hereinafter referred to as a “drug-delivery radiator”. The vibrator is positioned in or in a neighborhood of the target region and excited to vibrate to separate the drug and/or the particles comprising the drug from the vibrator and propel the drug to the target region.

Hereinafter, the word “drug” is used to indicate a drug and/or particles comprising the drug when discussing adhering the drug and/or particles comprising the drug to a drug-delivery vibrator or dispersing the drug and/or particles comprising the drug by exciting the vibrator to vibrate in accordance with an embodiment of the invention.

In accordance with an embodiment of the invention, the drug-delivery radiator is coupled to a distal end of a lead wire, rod, or catheter wire, hereinafter referred to generically as a catheter wire. The radiator is, optionally, an elongate radiator having an axis that is an extension of the axis of the catheter wire. Optionally, the drug-delivery radiator is formed as an integral part of the catheter wire.

To deliver the drug to the target site of an organ, the catheter wire is inserted into the organ and positioned so that the radiator is located in, or close to, the target region. An acoustic transducer, optionally coupled to the catheter wire at a proximal end thereof external to the organ, generates at least one pulse of ultrasound energy that is transmitted via the catheter wire to the radiator. Upon reaching the radiator, the at least one pulse of ultrasound energy generates vibrations therein that detach particles comprising the drug, and/or particles of the drug, from the radiator and disperse the particles into the target region.

According to an aspect of some embodiments of the invention, the ultrasonic radiator comprises a relatively long thin, optionally solid, ultrasonic “horn”. The horn is shaped so that acoustic energy transmitted to the horn along the catheter wire generate vibrations in the horn that are effective in dispersing the drug off and away from the horn in desired directions.

In some embodiments of the invention, acoustic energy transmitted along the catheter wire is transmitted as longitudinal acoustic waves. Generally, to provide effective drug dispersion it is advantageous to convert a portion of the received energy into transverse (with respect to the radiator's axis) vibrations of the radiator. Transverse vibrations transmit kinetic energy to the drug that tends to propel the drug radially away from the radiator. The inventors have found that a radiator having a cross section perpendicular to the horn's axis that undergoes relatively abrupt changes as a function of position along the axis has a tendency to convert longitudinal acoustic waves to transverse vibrations of the radiator.

According to an aspect of some embodiments of the invention, the radiator is formed as a spring shaped coil of wire. Optionally, the coils of the spring radiator are circular spirals.

An aspect of some embodiments of the invention, relates to enveloping an ultrasonic drug-delivery radiator with an “isolation jacket” having “exit” ports formed therein. In accordance with an embodiment of the invention, the jacket is filled with an “isolation liquid” that moderates influence of tissue in which the vibrator is positioned on the vibrator.

The inventors have determined that when a drug-delivery vibrator is inserted into a soft tissue, such as for example brain tissue, contact between the vibrator and surrounding soft tissue damps vibrations in the radiator. Damping may be so strong as to lower the Q of the vibrator to such an extent that the vibrator is not readily excited to vibrate with sufficient energy to disperse a drug effectively. The isolation jacket and liquid enable the vibrator to be effectively excited to vibrate and disperse a drug when positioned in soft tissue.

When the vibrator is inserted into the soft tissue, the isolation jacket is filled with the isolation liquid. The integrity of the soft tissue substantially seals the jacket's exit ports against egress of the isolation liquid from the jacket. As a result, the vibrator is “isolated” from the surrounding tissue in an environment that does not substantially affect the vibrator's Q and the vibrator is relatively easily and effectively excited to vibrate with sufficient energy to dislodge and propel the drug away from the vibrator. However, whereas the surrounding tissue effectively seals the jacket against egress of the isolation liquid, it does not prevent the propelled drug particles from exiting the jacket through the exit ports and lodging in surrounding tissue. The isolation jacket and its operation, in accordance with an embodiment of the invention, thus enable the vibrator effectively to deliver the drug to a region of soft tissue.

According to an aspect of some embodiments of the invention a drug-delivery radiator is expandable. In some embodiments of the invention, a drug-delivery radiator is “transported” to a site to which it is desired to deliver a drug in a compressed state so that it occupies a relatively small volume during transport. At the site, the drug-delivery radiator is expanded to better conform to dimensions of the site.

In some embodiments of the invention, an expandable drug-delivery radiator comprises a spring shaped radiator. The spring radiator is delivered to a site with its coils compressed inside a catheter. When pushed out of the catheter its coils expand. In some embodiments of the invention, an expandable drug-delivery radiator is formed similarly to a vascular stent and is expanded using technology similar to that used to expand a stent. After an expandable drug-delivery radiator in accordance with an embodiment of the invention is used to disperse a drug to a site it is retracted into the catheter which was used to transport the radiator and removed from the site with the catheter.

According to an aspect of some embodiments of the invention, at least one characteristic of the at least one pulse of ultrasound is controlled to control dispersion of the drug away from a drug-delivery radiator.

According to an aspect of an embodiment of the invention, the at least one characteristic is controlled so that an amount of energy deposited in tissue in the target region and in the dispersed drug by the at least one pulse does not damage the tissue or the drug. The inventors have determined that energy in the at least one pulse can be controlled so that, in general, the drug may substantially completely be removed from the radiator and dispersed without damaging the drug or tissue in the target region.

In some embodiments of the invention the at least one characteristic is controlled to control the kinetic energy with which particles of the drug are dispersed away from the radiator. For example, in an embodiment of the invention shape, amplitude, and/or frequency of the at least one pulse and/or a number of pulses comprised in the at least one pulse may be controlled to control the kinetic energy. By controlling the dispersal kinetic energy of drug particles dispersed from the spring radiator, a manner in which the drug particles are dispersed in tissue in the region and a dispersal range may be controlled.

According to an aspect of some embodiments of the invention, the at least one characteristic is controlled to stimulate vibrations in the radiator so that the particles dispersed off and away from the radiator into the target region have relatively high kinetic energy. In high kinetic energy dispersion, the drug particles generally have kinetic energy sufficient to penetrate cellular membranes in the target region.

According to an aspect of some embodiments of the invention, the at least one characteristic is controlled to stimulate vibrations in the radiator so that the particles dispersed have relatively low kinetic energy. In low kinetic energy dispersion, kinetic energy of dispersed drug particles is generally not sufficient for the particles to penetrate cell membranes and the drug particles are substantially constrained to move through interstitial liquid in the tissue region.

In some embodiments of the invention, dispersion of a drug is enhanced by sonophoresis. Optionally, sonophoresis is provided by controlling a drug-delivery radiator in accordance with an embodiment of the invention to vibrate at a frequency different from that used to disperse the drug off from the radiator.

Because a drug dispersed by a radiator in accordance with an embodiment of the invention is in solid form and direction and kinetic energy of dispersal of solid drug particles may be controlled, delivery and containment of the drug to a given target site can generally be controlled more accurately than in prior art drug-delivery systems. In addition, since the drug is not delivered systemically it is, optionally, not encapsulated or embedded in a hydrophilic material. Dosage of the drug administered to a patient may therefore not be limited by toxicity of a toxic hydrophilic embedding or encapsulating material.

There is therefore provide in accordance with an embodiment of the invention, apparatus for delivering a drug to a target site of a body comprising: a dispersing member adapted to vibrate when acoustically excited; a source of acoustic energy controllable to couple acoustic energy to the dispersing member to excite it to vibrate; and a drug adhered to the dispersing member so that when the acoustic source excites the dispersing member, the drug is dispersed therefrom.

Optionally, the dispersing member comprises an elongate body having an axis along its long direction. Additionally or alternatively, the dispersing member is characterized by relatively abrupt changes in its cross section perpendicular to the axis as a function of position along the axis.

In some embodiments of the invention, the dispersing member comprises a plurality of relatively large cross section regions separated by relatively small cross section regions. Optionally, the relatively large cross section regions have chamfered edges.

In some embodiments of the invention, the dispersing member comprises a plurality of cone shaped sections having relatively small first ends and relatively large second ends. Optionally, the first ends face a same direction. Optionally, the size of the cone shaped sections decrease as a function of distance along the dispersing member axis in the direction along which the first ends face.

In some embodiments of the invention, the dispersing member has a spiral screw shape.

In some embodiments of the invention, the dispersing member is integrally formed as a portion of a catheter wire. Optionally the apparatus comprises a catheter that comprises the catheter wire.

In some embodiments of the invention, the dispersing member comprises a spring having at least one coil formed from a wire and an axis. Optionally, the at least one coil comprises a plurality of coils. In some embodiments of the invention, all of the coils have a same size. In some embodiments of the invention, adjacent coils have different size. Additionally or alternatively, non-adjacent coils optionally have a same size.

In some embodiments of the invention, the coils comprise at least one relatively large first coil and at least one relatively large second coil and at least one intermediate coil smaller than the at least one first and at least one second coil located between them. Optionally the apparatus comprises a barrier adhered between the at least one first coil and the at least one second coil. Optionally, the barrier forms a surface having a lumen in which the at least one intermediate coil is located.

In some embodiments of the invention, the spring has a tapered shape in which the size of its coils decrease along a direction from a first end of the spring to a second end of the spring. In some embodiments of the invention, the coils have a constant pitch. In some embodiments of the invention, all coils have a same shape. In some embodiments of the invention, a coil of the at least one coil is circular.

In some embodiments of the invention, the dispersing member is integrally formed as a portion of a catheter wire. In some embodiments of the invention, the apparatus comprises a catheter that comprises the catheter wire.

In some embodiments of the invention, the coils of the spring are expandable. Optionally, the apparatus comprises a housing in which the spring may be housed with its coils compressed and from which it may be removed enabling the coils to expand. Optionally, the housing comprises ridges that are substantially parallel to the axis of the spring dispersing member and contact at least some of the compressed coils when the dispersing member is housed in the housing. Additionally or alternatively, the apparatus comprises a catheter wherein the housing comprises a portion of the catheter. In some embodiments of the invention, the dispersing member is integrally formed as a portion of a catheter wire.

In some embodiments of the invention, the dispersing member has a stent-like configuration. Optionally, the stent-like configuration has a compressed and an expanded state. Optionally the apparatus comprises a housing in which the dispersing member may be housed in its compressed state and from which it may be removed and changed into its expanded state.

In some embodiments of the invention, the apparatus comprises a jacket in which the dispersing member is positioned that has at least one exit port formed therein through which particles of the substance dispersed by the dispersing member exit. Optionally, the jacket is filled with a liquid, which when the dispersing member is positioned in the site or a neighborhood thereof, protects the dispersing member from contact with material at the site or in the neighborhood.

In some embodiments of the invention, the source of acoustic energy couples at least one pulse of acoustic energy to the dispersing member and controls at least one characteristic of the at least one acoustic pulse to control dispersion of the substance. Optionally, the acoustic source controls the at least one characteristic to control kinetic energy of particles of the substance dispersed from the dispersing member. Additionally or alternatively, the at least one characteristic comprises amplitude of the at least one acoustic pulse. In some embodiments of the invention, the at least one characteristic comprises frequency of the at least one acoustic pulse.

There is further provided in accordance with an embodiment of the invention, apparatus for delivering a drug in a neighborhood of a target site of a body to the site comprising: a dispersing member comprising at least one coil formed from a wire; and a source of acoustic energy controllable to couple acoustic energy to the dispersing member to excite it to vibrate; wherein when the drug and the dispersing member are located in a neighborhood of the site and the acoustic source excites the dispersing member, the dispersing member transmits acoustic waves that tend to propel the substance to the site.

There is further provided in accordance with an embodiment of the invention, apparatus for delivering a drug in a neighborhood of a target site of a body to the site comprising: a dispersing member comprising an elongate screw shaped body; and a source of acoustic energy controllable to couple acoustic energy to the dispersing member to excite it to vibrate; wherein when the drug and the dispersing member are located in a neighborhood of the site and the acoustic source excites the dispersing member, the dispersing member transmits acoustic waves that tend to propel the substance to the site.

There is further provided in accordance with an embodiment of the invention, apparatus for delivering a drug in a neighborhood of a target site of a body to the site comprising: a dispersing member comprising an elongate body having an axis and a cross section perpendicular to the axis that changes relatively abruptly as a function of position along the axis; and a source of acoustic energy controllable to couple acoustic energy to the dispersing member to excite it to vibrate; wherein when the drug and the dispersing member are located in a neighborhood of the site and the acoustic source excites the dispersing member, the dispersing member transmits acoustic waves that tend to propel the substance to the site.

There is further provided in accordance with an embodiment of the invention, apparatus for delivering a drug in a neighborhood of a target site of a body to the site comprising: an expandable dispersing member having a compressed and an expanded state; a source of acoustic energy controllable to couple acoustic energy to the dispersing member to excite it to vibrate; wherein wherein the drug is located in a neighborhood of the site and the dispersing member is transported to the neighborhood in the compressed state and at the neighborhood is transformed to its expanded state and when the acoustic source excites the dispersing member in the expanded state the dispersing member transmits acoustic waves that tend to propel the drug to the site.

There is further provided in accordance with an embodiment of the invention, a method of delivering a drug to a target site of a body comprising: providing a dispersing member adapted to vibrate when acoustically excited; adhering a drug to the dispersing member so that when the excited to vibrate the drug is dispersed therefrom; positioning the dispersing member at the site or a neighborhood thereof; and acoustically exciting the dispersing member. Optionally, providing a dispersing member comprises providing an expandable dispersing member having a compressed and an expanded state. Optionally, positioning the dispersing member comprises transporting the dispersing member to the site or a neighborhood thereof in the compressed state. Optionally, acoustically exciting the dispersing member comprises transforming the dispersing member to its expanded state at the site or the neighborhood thereof.

### BRIEF DESCRIPTION OF FIGURES

Non-limiting examples of embodiments of the present invention are described below with reference to figures attached hereto, which are listed following this paragraph. In the figures, identical structures, elements or parts that appear in more than one figure are generally labeled with a same numeral in all the figures in which they appear. Dimensions of components and features shown in the figures are chosen for convenience and clarity of presentation and are not necessarily shown to scale.

FIGS. 1A-1E schematically show an ultrasonic drug-delivery horn, in accordance with an embodiment of the invention;

FIGS. 2A-2C schematically show the drug-delivery ultrasonic horn shown in

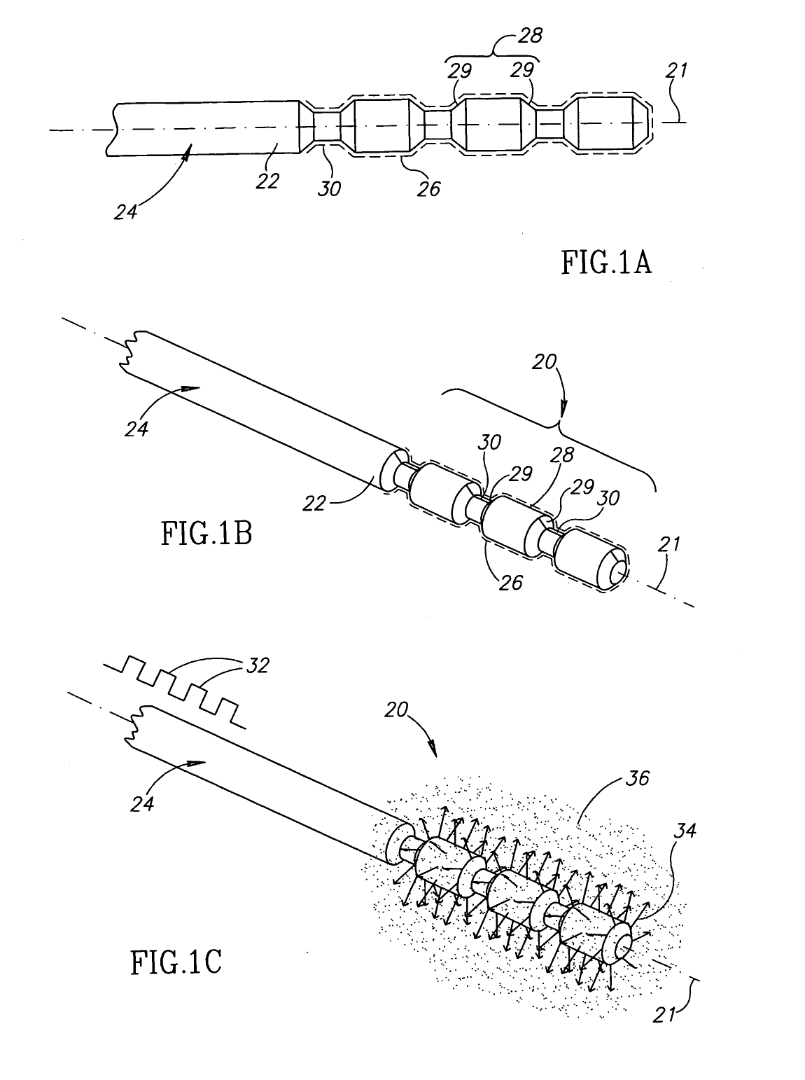

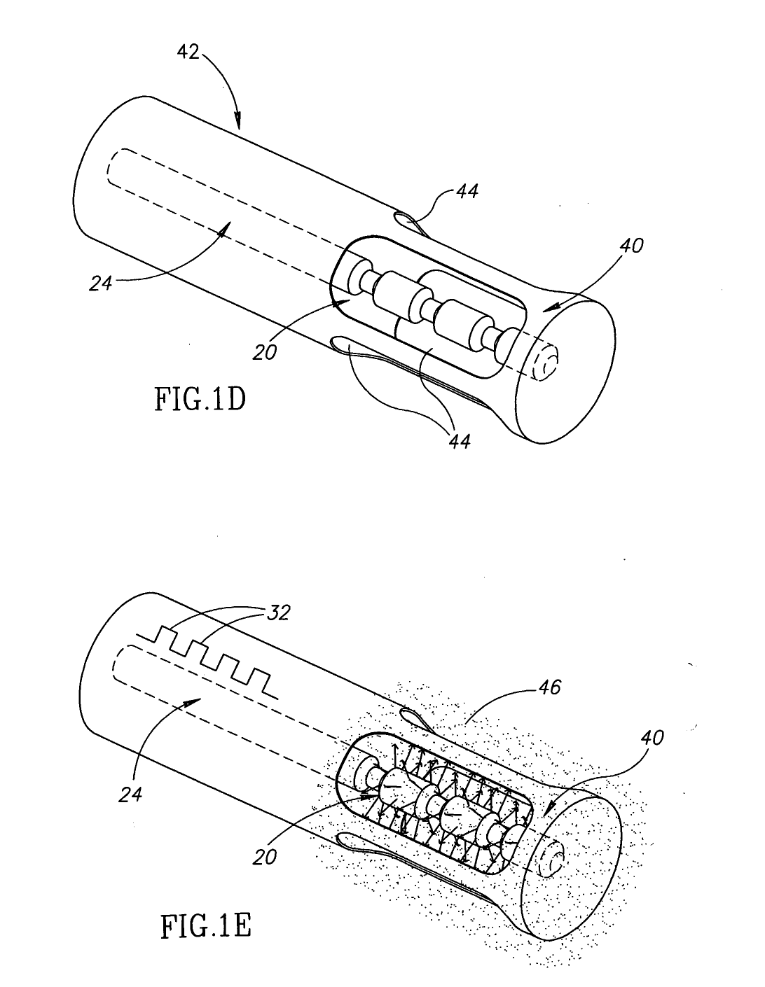

FIGS. 1A-1E being used to deliver an anticancer drug to a tumorous site in the brain of a patient, in accordance with embodiments of the present invention;

FIGS. 3A and 3B schematically show two other drug-delivery ultrasonic horn, in accordance with an embodiment of the present invention;

FIGS. 4A-4D schematically show a spring shaped ultrasonic drug-delivery vibrator, being used to deliver, by way of example, an anti-restenosis drug to a region of an artery;

FIGS. 5A-5C schematically show spring drug-delivery radiators having different shapes, in accordance with embodiments of the present invention; and

FIGS. 6A-6D schematically show an expandable drug-delivery radiator being used to deliver a drug to a site, in accordance with an embodiment of the present invention.

### DETAILED DESCRIPTION OF EXEMPLARY EMBODIMENTS

FIGS. 1A-1E schematically show an ultrasonic drug-delivery radiator in the shape of an elongate ultrasonic horn 20 having an axis 21, in accordance with an embodiment of the present invention. FIGS. 1A and 1B schematically show a longitudinal cross section of horn 20 and a perspective view of the horn respectively. Horn 20 is coupled to a distal end 22 of a catheter wire 24, only a portion of which is shown. Optionally, horn 20 is formed as an integral part of catheter wire 24 using any of many different methods, such as for example stamping and/or machining, known in the art.

A drug, represented by a dashed line 26, is adhered to surface regions of horn 20. In some embodiments of the invention, the drug is adhered to horn 20 using an electrodeposition process. In some embodiments of the invention, the drug is adhered by wetting the horn with an appropriate solution or dispersion containing the drug and then drying the horn.

Ultrasonic energy to excite horn 20 to vibrate and disperse drug 26 is optionally transmitted to horn 20 via catheter wire 24 in the form of at least one pulse of “longitudinal ultrasound”. To convert the at least one longitudinal pulse to transverse vibrations of horn 20, that tend to dislodge and propel particles of drug 26 radially away form horn 20, optionally, the horn is formed so that its cross section varies relatively abruptly along its length. The inventors have found that a horn having a relatively abruptly varying cross section tends to convert longitudinal waves to transverse vibrations of the horn. The inventors speculate that the abruptly changing cross section generates reflections and possibly diffraction of longitudinal waves that operate to convert a portion of the longitudinal acoustic energy to transverse vibrations. Optionally, horn 20 comprises a plurality of “bulbs” 28, optionally having chamfered edges 29, separated by connecting nubs 30. Optionally, the plurality of bulbs comprises three bulbs 28.

FIG. 1C schematically shows a plurality of ultrasonic pulses 32 propagating along catheter wire 24 to horn 20 and generating vibrations in the horn, which propel drug 26 off and away from the horn. The inventors have found that when excited to vibrate a horn, in accordance with an embodiment of the invention, having a shape similar to that shown in FIGS. 1A-1C disperses drug 26 in directions indicated by arrows 34 into a substantially cylindrical “dispersion volume” indicated by a stippled region 36. Dispersion of drug 26 into dispersion volume 36 is substantially uniform as a function of azimuth angle relative to axis 21 of horn 20 and length along the horn.

By way of numerical example, a horn radiator in accordance with an embodiment of the invention similar to horn 20, may be formed as an integral part of a catheter wire 24 optionally having a diameter equal to about 1 mm. Each bulb 28 optionally has a diameter about equal to that of wire 24 and a length of about 1.1 mm and each connecting nub 30 has a diameter equal to about 0.6 mm and a length equal to about 0.5 mm. For a three bulb horn such as shown in FIGS. 1A-1E the length of the horn is therefore, optionally, about 4.95 mm. (Three sets of bulbs 28 and nubs 30 are optionally about 4.8 mm long and an additional chamfered edge 29 at the junction of radiator 20 and catheter wire 24 having an axial extent equal to about 0.15 mm results in an overall length of about 4.95 mm). Optionally, horn 20 is formed from a Titanium alloy approved for medical applications. Optionally the alloy is a Titanium alloy comprising about 6% Aluminum and about 4% Vanadium (approved by the FDA and used for medical implants) or a Titanium alloy comprising about 15% Vanadium, 3% Chromium, 3% Nickel and about 3% Aluminum.

Dimensions for a horn, in accordance with an embodiment of the invention, similar to horn 20 other than those noted above are of course possible and can be advantageous. For example, bulbs 28 may have diameters equal to about 2 mm and horn 20 an overall length of 10 or 20 mm. In general, the size of a horn, such as horn 20 may be adapted to size requirements of a site in which it is to be used and dimensional constraints of a manner in which the horn is transported to and positioned in the site.

In some embodiments of the invention, horn 20 is enveloped in a protective isolation jacket 40, which is schematically shown in FIG. 1D. Optionally, isolation jacket 40 is part of a catheter 42 that houses catheter wire 24. Jacket 40 is formed with a plurality of exit ports 44. By way of example, jacket 40 is shown formed with four exits ports symmetrically positioned around the circumference of jacket 40.

The inventors have found that when a horn similar to horn 20 enveloped in a jacket, in accordance with an embodiment of the invention, is positioned in a soft tissue and the jacket filled with an isolation liquid, the horn can be effectively excited to vibrate and disperse a drug into the tissue. The inventors have also determined that the size of exit ports 44 of jacket 40 can be made sufficiently large and close to each other so a size and shape of a dispersion volume of horn 20 is not substantially affected by the jacket and yet the isolation liquid does not flow out of the jacket. The liquid is prevented from leaking out of the isolation jacket by the soft tissue in which the radiator is positioned, which operates to seal the exit ports against egress of the liquid. By way of numerical example, isolation jacket 40 optionally has a wall thickness of about 0.3 mm, an outer diameter of about 2.1 mm, and ports 44 a length of about 6 mm millimeters and a width of about 0.8 mm.

As in the case of dimensions of a horn in accordance with an embodiment of the invention, dimensions of a jacket may be different from those noted above. Size of a jacket, such as jacket 40, are in general, adaptable to dimensions of a horn with which it is to be used and size requirements of a site in which the horn and jacket are used and dimensional constraints of a manner in which the horn and jacket are transported to and positioned in the site.

In some embodiments of the invention exit ports are configured to configure to configure a dispersion volume of a drug-delivery radiator. For example, a jacket similar to jacket 40 may have only one exit port so that a radiator, such as horn 20, disperses a drug in a limited azimuthal direction. A horn enveloped in such a jacket may be oriented to disperse a drug in desired azimuthal direction in tissue in which the horn and jacket are located.

FIG. 1E schematically shows a plurality of ultrasound pulses 32 being transmitted to horn 20 shown in FIG. 1D and drug 26 being dispersed in a dispersion volume 46 substantially the same as that of dispersion volume 36 schematically shown in FIG. 1B.

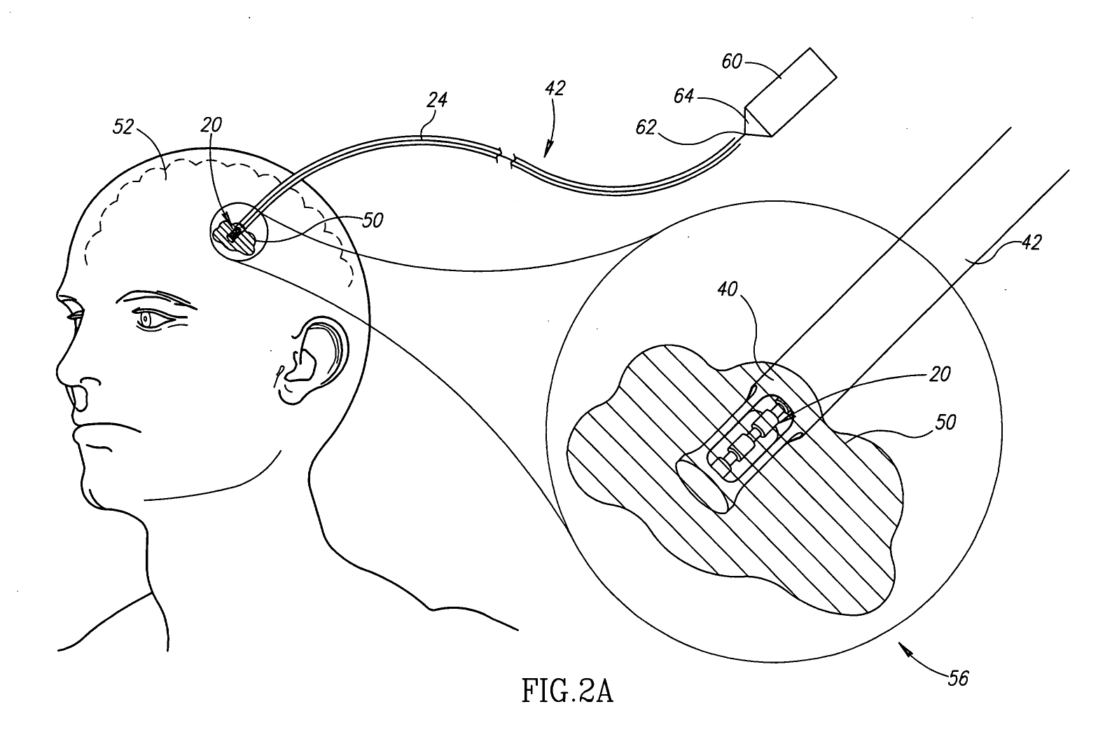

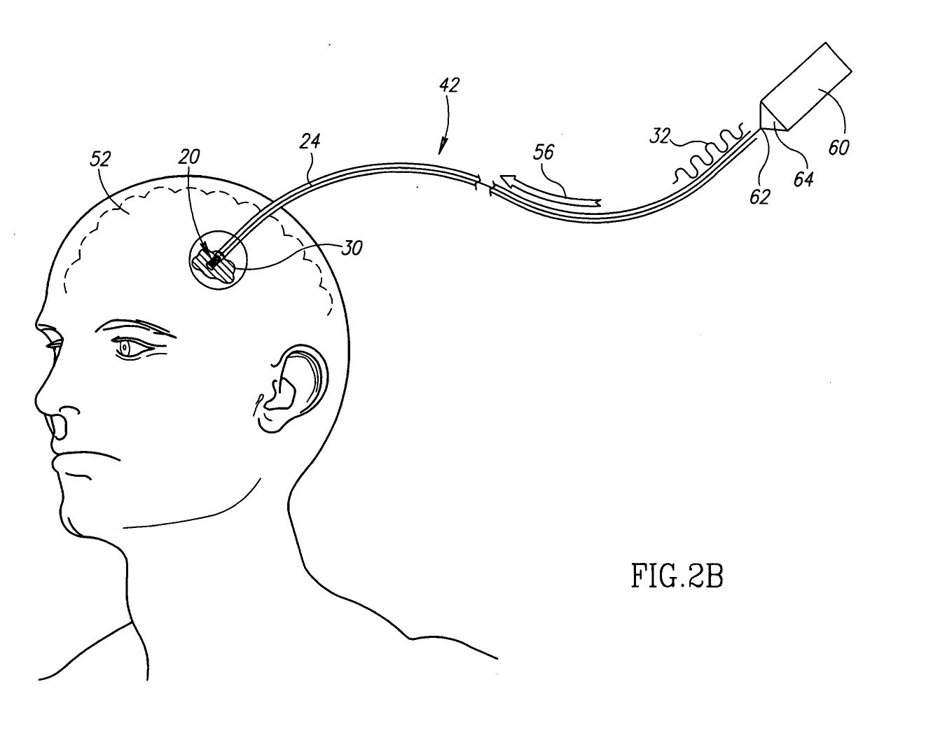

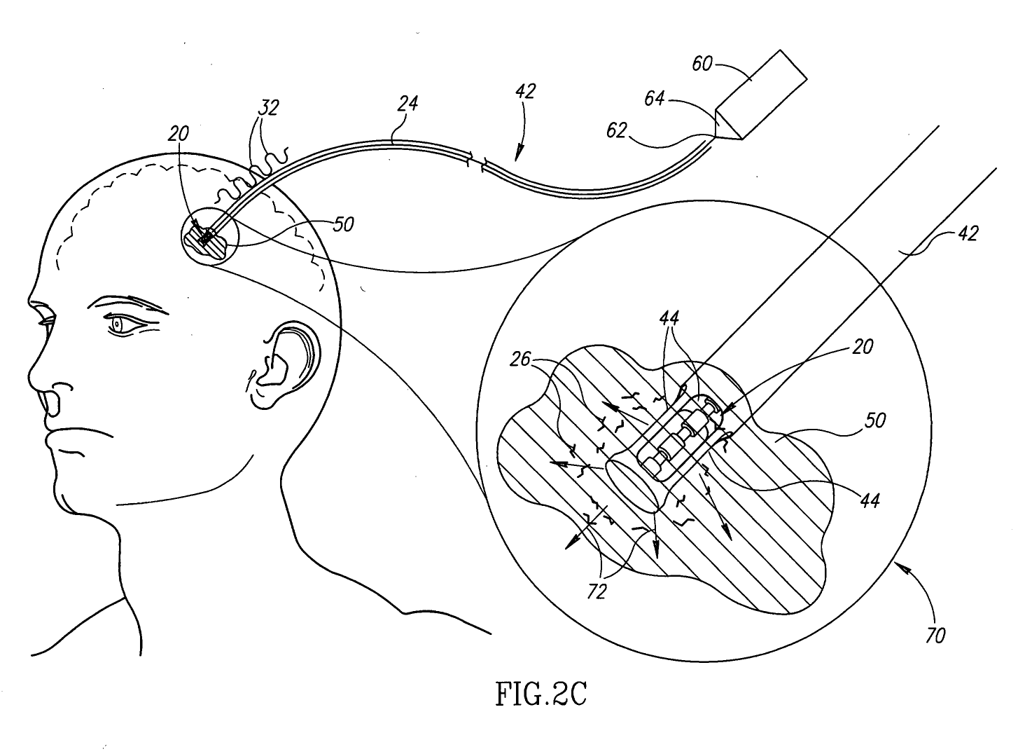

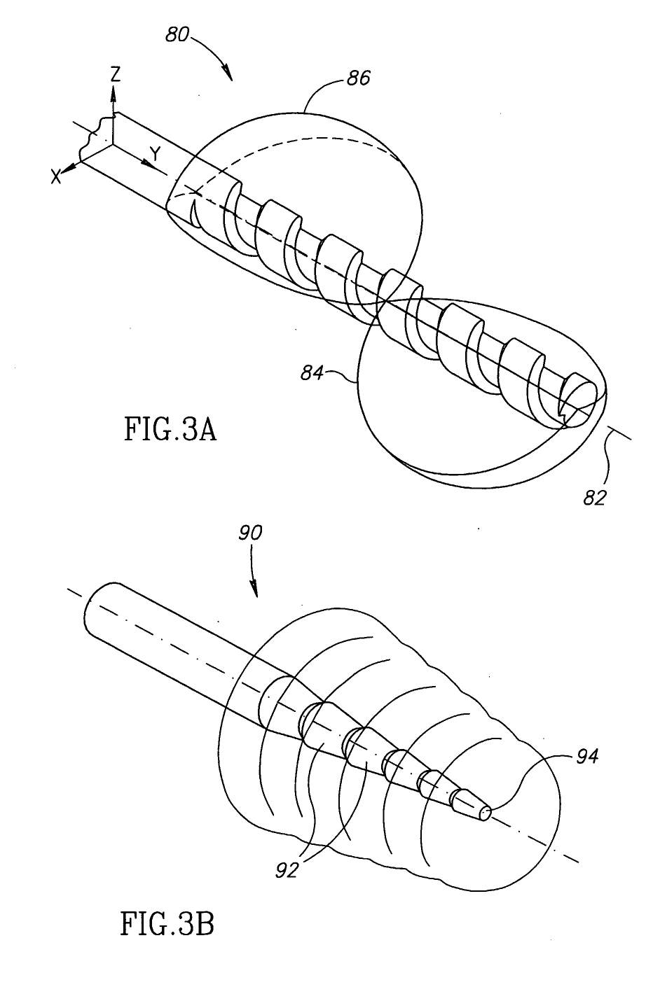

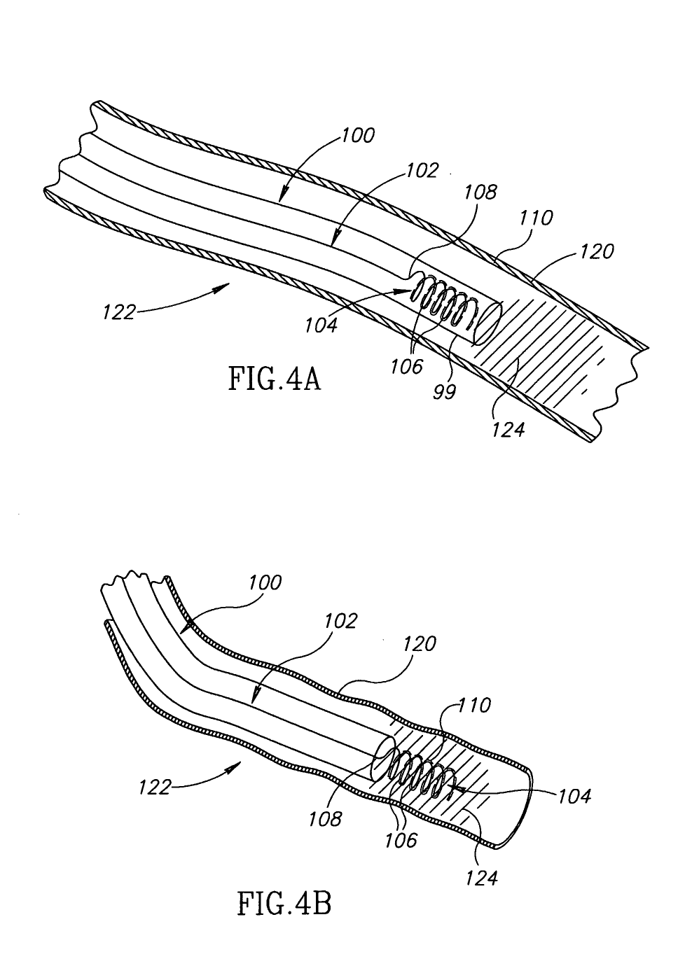

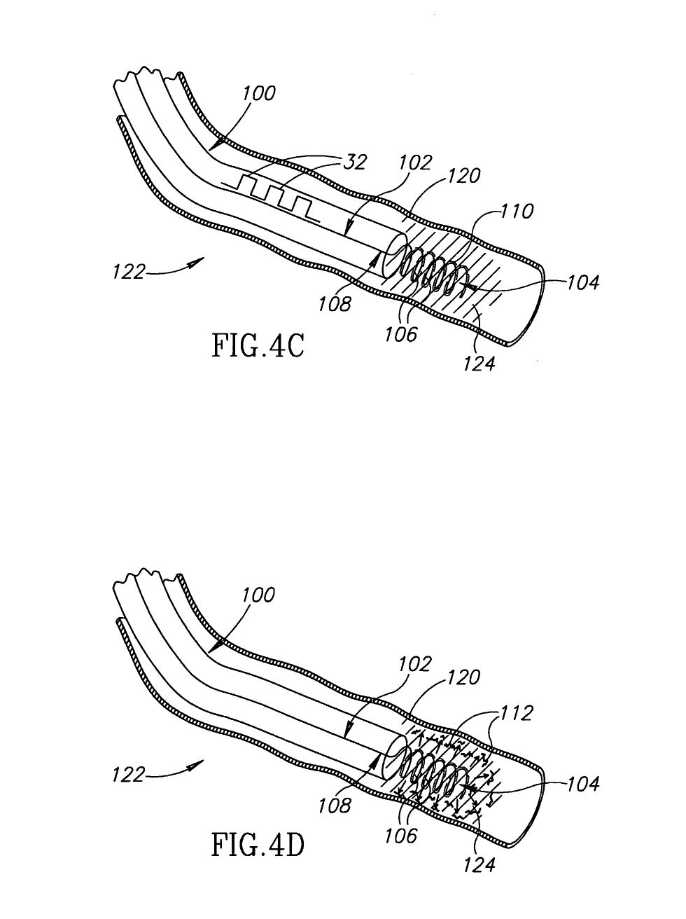

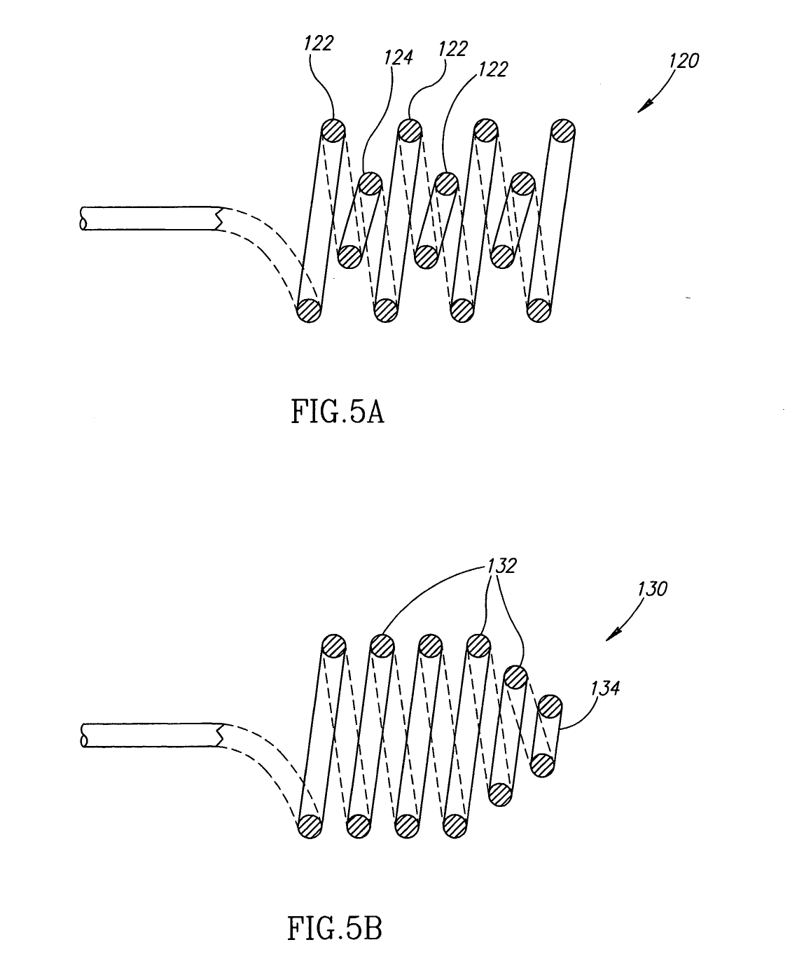

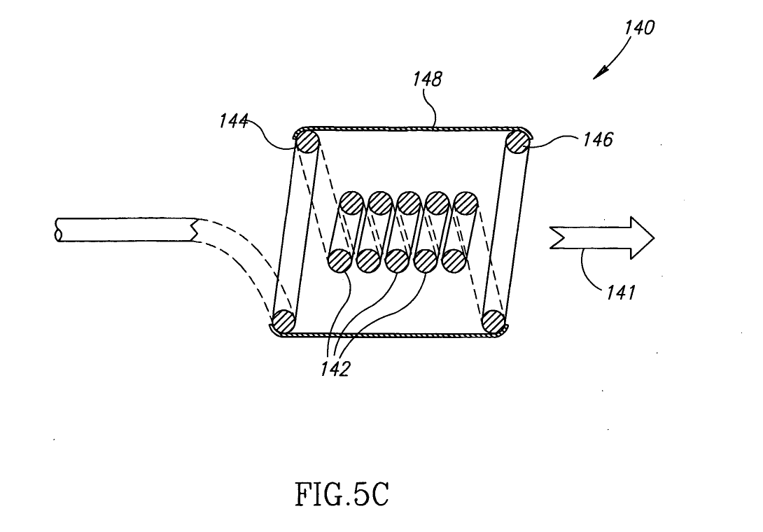

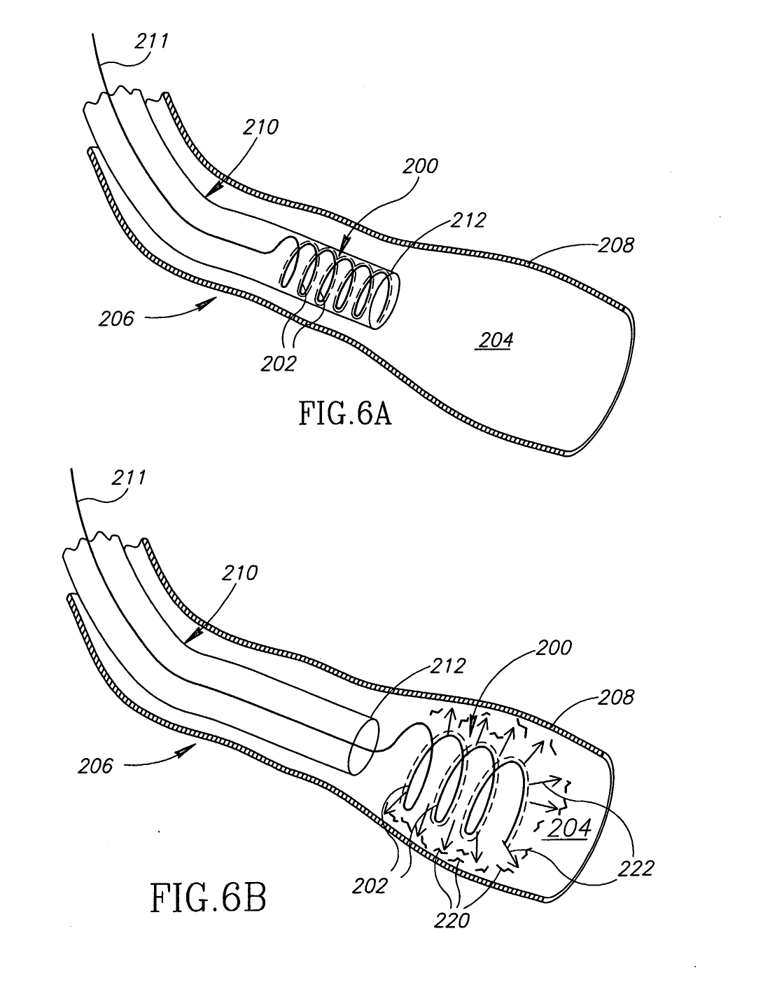

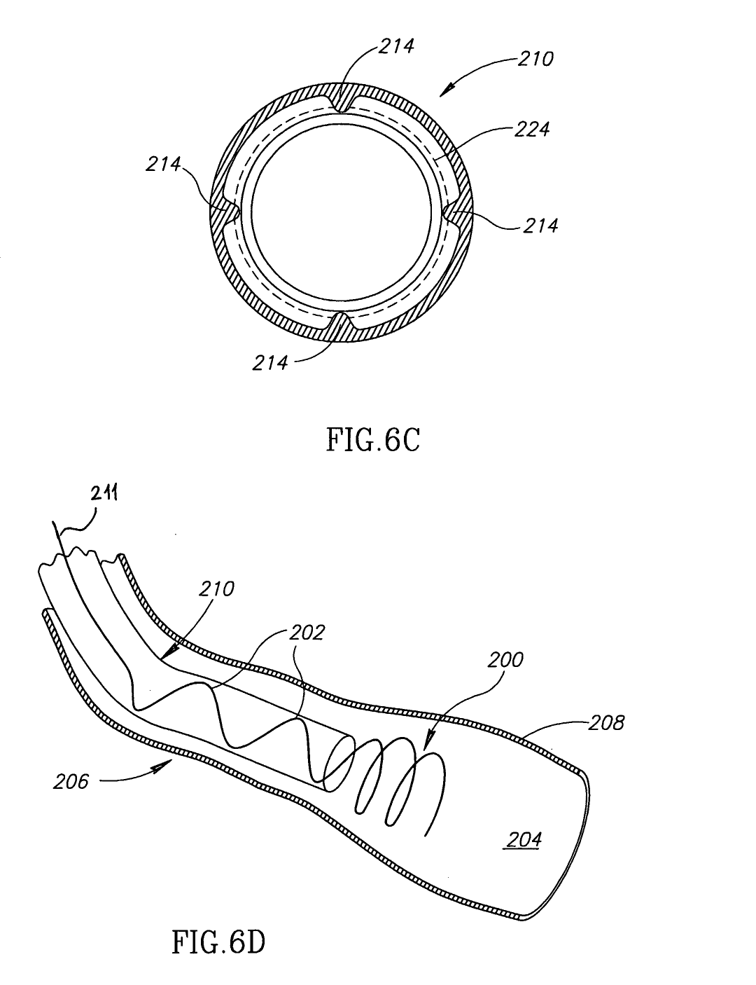

FIGS. 2A-2C schematically show ultrasonic drug-delivery horn 20 shown in FIGS. 1A-1E being used, by way of example, to deliver an anti-cancer drug 26, to a tumorous site indicated by a shaded region 50 of a patient's brain 52, in accordance with an embodiment of the invention. Anticancer drug 26 may, by way of example be Paclitaxel, Mitomycin, Doxorubicin, MTX or BCNU (Carmustine, 1,3-bis(2-chloroethyl)-1-nitrosourea) microencapsulated or embedded in, or on the surface of, suitable nanoparticles. An acoustic transducer 60 is optionally coupled to a proximal end 62 of catheter wire 24 and is controllable to generate at least one pulse of ultrasonic energy that is transmitted along the catheter wire to horn 20. Optionally, acoustic transducer 60 is coupled to catheter wire 24, using methods known in the art, by an impedance matching adapter 64 that improves efficiency with which acoustic energy in the at least one pulse is coupled to the catheter wire.

FIG. 2A schematically shows catheter 42 just after it has been inserted into the patient's brain using methods known in the art so that drug-delivery horn 20 is located close to or within tumorous site 50 and is ready to be activated to disperse drug 26 to the site. Jacket 40 is filled with an isolation liquid that prevents tissue in tumorous site 50 from making direct contact with horn 20 and damping vibrations in the horn when the horn is excited to vibrate. Optionally the isolation liquid is glycerol or a saline solution. An inset 56 schematically shows an enlarged view of horn 20 located in tumorous region 50.

FIG. 2B schematically shows acoustic transducer 60 after it has been controlled to transmit, optionally, a train of ultrasound pulses 32, into catheter wire 24 so that the train propagates along the catheter wire to ultrasound horn 20 in a direction indicated by block arrow 58.

FIG. 2C schematically shows horn 20 at a time at which pulses 32 reach the horn and generate vibrations therein that disperse drug 26 to tumorous site 50, in accordance with an embodiment of the invention. An inset 70 schematically shows dispersal of drug 26 represented by particles of the drug dispersing outward away from horn 20 and through exit ports 44 of jacket 40 in directions indicated by arrows 72.

According to an aspect of an embodiment of the invention at least one characteristic of ultrasound pulses 32 is controlled to control dispersion of drug 26 away from horn 20. In some embodiments of the invention, the at least one characteristic is controlled to control the kinetic energy with which particles of drug 26 are dispersed away from the horn. For example, assume that site 50 is relatively large and that it is desired that particles of drug 26 penetrate walls of cancerous cells in site 50 in order to lodge in the cells. Kinetic energy of particles of drug 26 dispersed by horn 20 should therefore be relatively large. In accordance with an embodiment of the invention, to provide the required kinetic energy amplitude and/or repetition frequency of pulses 32 are controlled to stimulate appropriate vibrations in horn 20.

It is noted that kinetic energy and therefore range of particles of a given drug dispersed by a drug-delivery radiator in accordance with an embodiment of the present invention in general depends on a number of different variables. For example, the kinetic energy will in general depend inter alia on energy required to break bonds that bind the drug to the drug-delivery radiator and acoustic impedance of material from which the radiator is formed and the environment, e.g. tissue or isolation liquid, in which the radiator is located. Dispersion range of the particles in the tissue and ability of the particles to penetrate cell walls in the tissue will also in general depend inter alia on characteristics of the tissue and form of encapsulation of the drug. Suitable look up tables (LUTs) may be provided a user of a drug-delivery radiator in accordance with an embodiment of the invention to guide operation of the radiator. For a given radiator configuration, a LUT may relate for example, target tissue type, drug type, dispersion range, cell wall penetration and characteristics of ultrasound pulses used to excite vibrations in the radiator. Optionally, the LUTs are generated from experimental data.

Shapes of ultrasonic drug-delivery horns different from that shown in FIGS. 1A-2C are possible and may provide advantageous characteristics. FIG. 3A schematically shows a drug-delivery horn 80 having an axis 82 and a spiral, right-hand screw shape that disperses a drug in a dispersion volume that is different from the cylindrical volume shown in FIGS. 1B and 1D. Horn 80 tends to disperse particles adhered to it in a dispersion volume having two dispersion lobes located substantially on opposite sides of horn 80, a forward lobe 84 at the front end of the horn and a back lobe 86 at the back of the horn 80. Each lobe has a maximum, fan shaped cross-section perpendicular to axis 82 of horn substantially at the center of the lobe.

A horn similar to horn 80 can be used to disperse a drug in a desired azimuthal direction relative to axis 82. For example, it is possible to coat only the back or front end of horn 80 so that the horn disperses a drug adhered to its surface into only one of its dispersion lobes. It is of course also possible to coat horn 80 with a drug all along its length and envelope horn 80 in an isolation jacket having at least one slot shaped exit port parallel to axis 82 formed on only one side of the jacket. The horn may be positioned relative to the exit port so that when excited to vibrate, a region of the horn from which a dispersion lobe originates is opposite the port and drug particles dispersed into the lobe pass through the exit port to be delivered to a tissue region. The probe may be rotated to position first one and then the other of the lobes opposite the exit port.

FIG. 3B schematically shows another a drug-delivery horn 90, in accordance with an embodiment of the invention. Horn 90 comprises a plurality of similar, cone shaped sections 92 that point toward a front end 94 of the horn. Optionally, size of the cone sections decrease with proximity to front end 94 of the horn. Horn 90 tends to disperse particles adhered to its surface in a forward direction within a substantially cylindrically symmetric dispersion volume having a diameter that tends to become smaller toward front end 94 of the horn. Horn 90 may be used advantageously in situations for which it is desired to disperse a drug preferentially forward in an axial direction, such as when it is desired to deliver a thrombolytic agent to a thrombosis in a blood vessel.

As noted above, drug 26, which may for example be an anticancer drug, is optionally deposited on a drug-delivery radiator in accordance with an embodiment of the invention, such as exemplary horns 20, 80 or 90, by electrodeposition. Electrodeposition has a number of advantageous characteristics. It may generally be performed at room temperature, does not cause chemical reaction between the deposited drug and the surface of the horn and may be controlled to deposit a relatively accurately known quantity of the drug onto the radiator. Examples of anticancer drug that may be electrodeposited are BCNU (Carmustine, 1,3-bis(2-chloroethyl)-1-nitrosourea), Carboplatin (1,1-Cyclobutanedicarboxylatodiammine Platinum (II)) or Doxorubicin (14-hydroxy derivative of Daunorubicin), or for example paclitaxil encapsulated in iron oxide (IO) particles.

For electrodeposition of, by way of example the drug paclitaxil encapsulated in IO particles on a horn, in accordance with an embodiment of the invention, the surface of the horn is optionally first etched in a suitable acid solution such as a solution of nitric acid, hydrofluoric acid or a mix of acids. The etching removes oxides from the surface of the horn and activates the surface. For a relatively quick release of the drug from the horn when ultrasound pulses excite the horn, the drug is optionally electrodeposited directly onto the cleaned surface of the spring radiator. For a relatively slow release, a two to three micrometer thick, relatively uniform porous layer of a suitable oxide, such as for example Titanium oxide, to which the drug can readily adhere, may be formed on the surface by electrochemical oxidation using methods known in the art.

To deposit the drug on the treated surface of the horn, the horn is optionally immersed in a suspension that comprises from about 1% to about 20% by weight of the encapsulated drug suspended in an organic solvent, such as for example alcohol, ethanol, acetone or Isopropanol. Optionally, the encapsulating particles have a diameter of between about 10 nanometers to about 5 microns. Optionally, the suspension comprises between about 10⁻⁵ to about 10⁻³ grams per liter of a charge agent, such as cationic or anionic polyelectrolytes or a salt such as aluminum chloride.

During immersion, the encapsulating particles are removed from the suspension and adhered to the horn by maintaining the horn at a positive or negative voltage relative respectively to a suitable cathode or anode immersed in the suspension. Magnitude of the voltage is such as to generate an electric field between the horn and the anode or cathode that has a value in a range from about 1 volt/cm to about 200 volts/cm. For a positive voltage maintained on horn 90 the suspension optionally has a pH in a range from about 8 to about 9 and for a negative voltage the pH of the suspension is optionally from about 4 to about 6.

In some embodiments of the invention, an ultrasonic drug-delivery radiator has a shape of a spring. FIGS. 4A-4D schematically show a catheter 100 comprising a catheter wire 102 having an ultrasonic spring radiator 104 coupled to a distal end 108 of the catheter wire, in accordance with an embodiment of the invention. Spring radiator 104 comprises coils 106 to which a drug, represented by a dashed line 110, is deposited, optionally using an electrodeposition process. Optionally the electrodeposition process is similar to the electrodeposition process described above. By way of example, drug 110 is assumed to be an antirestenosis drug, such as paclitaxel, rapamycin or a corticosteroid, and in the figures, spring radiator 100 is shown being used to deliver the drug to walls 120 of a patient's blood vessel 122 in a region indicated by a shaded region 124 of the blood vessel.

FIG. 4A schematically shows catheter 100 just after it has been inserted into blood vessel 122 so that a distal end 99 of the catheter is located in a neighborhood of region 124. During insertion of catheter 100 into blood vessel 122, catheter wire 24 is in a retracted position in which spring radiator 100 is located inside the catheter to protect spring radiator 100 and drug 110 from damage that might occur as a result of contact with walls of 120 of blood vessel 122. In FIG. 1A spring radiator 104 is shown located retracted for protection, inside the catheter lumen.

Spring radiator 100 is optionally formed as an integral part of catheter wire 102, optionally has constant pitch and each coil 106 of the spring is optionally circular and has a same diameter. Optionally, the cross section of wire from which coils 106 are formed is circular. An acoustic transducer (not shown) is optionally coupled to a proximal end of catheter wire 102 outside of the patient's body and is controllable to generate at least one pulse of ultrasonic energy that is transmitted along the catheter wire to spring radiator 100. Optionally, the acoustic transducer is coupled to catheter wire 102, similarly to the manner in which acoustic transducer 60 shown in FIGS. 2A-2C is coupled to catheter wire 24.

FIG. 4B schematically shows catheter wire 102 moved to an active position in which spring radiator 100 protrudes from distal end 99 of catheter 100 in a neighborhood of region 106 and is ready to be activated to disperse drug 110 to walls 120 of blood vessel 122 in the region.

In FIG. 4C the acoustic transducer coupled to the proximal end of catheter wire 102 has been controlled to transmit, optionally, a train of ultrasound pulses 32, into catheter wire 102 that propagates along the catheter wire to spring radiator 104. When pulses 32 reach the spring radiator they generate vibrations in the radiator that tend to explosively detach drug 110 from surface regions of spring radiator 104 to which the drug is attached and propel the drug away from the spring and into blood vessel walls 120.

FIG. 4D schematically shows spring radiator 104 at a time at which pulses 32 reach the spring and vibrations generated by the pulses in the spring radiator disperse drug 110 to blood vessel walls 120 in region 124, in accordance with an embodiment of the invention. Dispersal of drug 110 is schematically represented by particles of the drug dispersing outward in directions indicated by arrows 112 away from spring radiator 104.

As in the case of drug-delivery ultrasonic horns in accordance with embodiments of the invention, at least one characteristic of ultrasound pulses 32 is controlled to control dispersion of drug 110 away from spring radiator 104. For the procedure schematically shown in FIGS. 4A-4D, kinetic energy of vibrations in spring radiator 104 is controlled so that drug particles dispersed by the radiator preferentially lodge in blood vessel walls 122.

A number of considerations influence the design of spring radiator 104, specifications for adhering drug 110 to regions of the spring's surface and specifications of ultrasound pulses 32 that energize the spring radiator so that it properly releases the drug.

It is noted that whereas ultrasound pulses 32 are substantially longitudinal pulses as they propagate along catheter wire 102, when they are incident on spring radiator 104 a portion of the energy in the pulses is converted to transverse vibrational energy. Conversion of longitudinal vibrational energy to transverse vibrational energy occurs as a result of reflection and refraction of pulses 32 at distal end 108 where spring radiator 104 and catheter wire 102 are joined and at the interface between the surface of the spring and tissue in region 124. Reflection and refraction of pulses 32 at the interface are a function of a difference in the acoustic impedance of tissue in region 124, in the instant case blood, and material from which spring 104 is formed. The dispersal of particles of drug 110 from spring radiator 104 is therefore a relatively complicated process to which, in general, both longitudinal and transverse vibrations contribute.

Longitudinal vibrations of the material from which spring radiator 104 is formed cause the radii of coils 106 to cyclically increase and decrease and the coils to displace back and forth axially along the length of the spring at the repetition frequency of pulses 32. As a result, longitudinal vibrations tend to impart kinetic energy to particles of drug 110 along tangents to coils 106 and also radially and axially. Transverse vibrations tend to disperse drug particles away from spring radiator 104 in directions perpendicular to the wire in a coil 106.

Spring radiator 104 is in general, as discussed below in a numerical example, a relatively small and delicate device and can be subject to substantial shear forces when excited by an ultrasound pulse such as ultrasound pulses 32. Therefore, the spring radiator should have sufficient strength so that shear forces generated in the radiator by ultrasound pulses 32 do not damage, and in particular do not break, the spring radiator.

By way of a numerical example, a spring radiator in accordance with an embodiment of the invention similar to spring radiator 104 may be formed as an integral part of a 1 mm diameter catheter wire 102. Optionally catheter wire 102 is formed from a Titanium alloy approved for medical applications that comprises about 6% Aluminum and about 4% Vanadium. Spring radiator 104 is optionally formed by cold drawing a section of catheter wire 102 at its distal end 108 to an optionally circular cross wire having diameter equal to about 0.3 mm. The cold drawn wire is then twisted into coils 106 using methods and devices known in the art. Optionally, spring radiator 104 has a length of about 6 mm and coils 106 have an outer diameter of about 1 mm and a pitch of about 1 coil/mm.

Dimensions of a spring radiator, in accordance with an embodiment of the invention, similar to spring radiator 104 other than those noted above are of course possible and can be advantageous. For example, a spring radiator may have 2 mm diameter coils, and/or a pitch of about 1 coil per 1.5 mm and/or an overall length of 20 mm. In general, the size of a spring radiator may be adapted to size requirements of a site in which it is to be used and dimensional constraints of a manner in which the horn is transported to and positioned in the site.

In the above discussion, by way of example, all coils 106 in spring radiator 104 are identical and pitch of the coils along the length of the spring radiator is substantially constant. Other spring configurations in accordance with an embodiment of the invention are possible and can be advantageous. FIG. 5A-5C schematically show spring radiators having different exemplary configurations in accordance with embodiments of the present invention.

FIG. 5A schematically shows a spring radiator 120 comprising coils having different diameters, in accordance with an embodiment of the invention. By way of example, spring radiator 120 comprises a coil configuration in which large diameter coils 122 alternate with small diameter coils 124. The shape of spring radiator 120 can be advantageous for maintaining integrity of a drug coating deposited on the spring radiator. When a spring radiator is introduced into a target region for which it is to be used to disperse a drug, surface regions of the spring will in general contact and rub against tissue in and near the target region. The rubbing tends to dislodge quantities of the drug deposited on the spring radiator surface. For the “alternating coil diameter” spring radiator 120, drug deposited on the small diameter coils 124 is protected from removal by abrasion by the large diameter coils 122 when the coil is introduced into a target region. To prevent unwanted deposition of the drug in tissue regions by abrasive removal of the drug from large coils 122 as spring radiator 120 is introduced into a target tissue region, the drug may be removed from the outer surfaces of the large coils before using the radiator.

FIG. 5B schematically shows a spring radiator 130, in accordance with an embodiment of the present invention, having a tapered shape in which the diameter of its coils 132 decrease with proximity to a free end 134 of the coil. The shape of spring radiator 130 can facilitate pushing the spring into a tissue region or threading the spring through a region of a blood vessel.

FIG. 5C shows a spring radiator 140 comprising a baffle that prevents dispersion of a drug in unwanted directions, in accordance with an embodiment of the present invention. Spring radiator 140 is used for dispersing a drug axially in a forward direction indicated by a block arrow 141 from the spring.

Spring radiator 140 comprises a plurality of intermediate diameter coils 142 sandwiched between at least one large “back-end” coil 144 and at least one large “front-end” coil 146. Following deposition of a drug on spring radiator 140 a “baffle” film 148 is formed that is anchored to and extends between at least one back-end coil 144 and at least one front-end coil 146. Baffle film 148 is optionally formed from a suitable polymer, such as for example PVC, and anchored to at least one back-end coil 144 and at least one front-end coil 146 using methods known in the art. For example, baffle film 148 may be bonded to the front and back end coils using a suitable bonding agent such as Auroro UV-S 2051 UV Curable Plastic Bonder marketed by Ellsworth Adhesives of the US.

When spring radiator 140 is excited by suitable ultrasound excitation pulses, baffle film 148 blocks drug particles adhered to spring radiator 140 that are propelled away from the spring radiator substantially laterally from being dispersed to tissue in which the radiator is positioned. As a result the drug is dispersed preferentially axially. A spring radiator in accordance with an embodiment of the invention similar to spring radiator 140 may be particularly advantageous for use in treating a thrombosis. The radiator may be threaded through the vascular system to the sight of a thrombosis and acoustically excited to “jet” a dose of a thrombolyte axially forward in a direction indicated by block arrow 141 and into the thrombosis, with relatively little exposure of healthy tissue to the thrombolyte.

Whereas the spring radiators, for example in FIGS. 5A-5C, are shown as being formed from wire having a circular cross sectional area, spring radiators in accordance with embodiments of the invention may be formed from wire having a cross section other than circular. For example, the cross section may be elliptical or rectangular.

The inventors have carried out experiments to test aspects of the operation of drug-delivery ultrasound radiators, in accordance with embodiments of the invention, in dispersing nanoparticles suitable for use as carriers of medication in brain tissue.

In one series of experiments, an ultrasonic horn similar to that shown in FIGS. 1A-2C was coated with iron oxide (IO) particles having diameters in a range from about 15 nm to about 20 nm. IO particles are optionally used with ultrasound radiators in accordance with embodiments of the invention as particulate delivery agents for drugs. IO nanoparticles are classified as biodegradable and may be coated with a suitable polymer, such as a starch, a silicone, dextran, albumin, poly-ethyleneglycol or PMMA (Poly(methyl methacrylate)), to which molecules of a drug to be delivered to a desired site can be coupled. A drug can also be loaded into the volume of IO particles during manufacture of the particles so that during degradation of the particles in a tissue in which they are located, the drug is released to the tissue.

After being coated with the IO nanoparticles, the horn, enveloped in an isolation jacket filled with an isolation liquid comprising a 0.25 mg/ml saline solution, was introduced into the center of the striatum of the brain of a male Fisher rat under general anesthesia. A power supply set at an output power of about 4 watts generated a two minute long train of 20 kHz ultrasound pulses to excite the horn to vibrate and disperse the IO particles into the brain tissue. The pulses had pulse widths of about two seconds and repetition rate of 20 pulses per minute. The procedure was repeated for each of a first group of male Fisher rats. A second group of rats underwent a similar procedure in which the horn was excited for 2 minutes by a train of ultrasound pulses having pulse widths of about 1 second and repetition rate of 30 pulses per minute generated at an output power of about 2 watts. Similar experiments were carried out for excitation periods of about 1 minute rather than 2 and for IO particles covered with a coating of dextran and having sizes from between about 50 nm to about 70 nm.

During post treatment observation the rats did not evidence abnormal behavior that might have resulted from the treatment and macroscopic and microscopic examinations of tissue specimens from the brains were negative for evidence of tissue damage, cyst formation or necrosis. MRI images indicated that for the first group of rats (excited at 4 watts by 2 second long pulses) the excited horn dispersed IO particles into an ellipsoidal region of brain tissue extending to a distance of about 5 mm from the axis of the horn and along the horn for a distance of about 8 mm. For the second group of rats (excited at about 2 watts by 1 second long pulses) IO particles were dispersed to a distance of about 2.5-3 mm. Shorter excitation periods of the horn resulted in dispersion to shorter distances. For the same excitation conditions, the larger IO particles (50 nm to about 70 nm) were dispersed to substantially same distances from the horn as the smaller IO particles.

Another set of experiments was performed in-vitro on cow brain tissue. For these experiments, the drug-delivery radiator was a spring radiator comprising 1 mm diameter coils, a pitch of about 1 coil/mm and an overall length of about 6 mm. For some of the experiments the spring was covered with 15-20 nm IO particles and for some with blue Polystyrene particles having a diameter of about 180 nm. For each of the different type of particles, the spring was sheathed in an isolation jacket filled with a saline solution isolation liquid and excited for excitation periods of 1, 2 and 3 minutes with 2 second long, 20 kHz ultrasound pulses, at a pulse repetition rate of about 20 pulses per second. The power supply generating the pulses operated at about 10 watts. Dispersion of particles was substantially independent of the particle size and for the excitation periods of 1, 2 and 3 minutes, particles were dispersed in the brain tissue to distance of about 10 mm, 15 mm and 25 mm respectively.

The ability to deposit a drug in tissue so that it remains there over a relatively long period of time was tested in another set of experiments. In the experiments, IO particles were dispersed in the brains of rats using methods and procedures similar to those described above. Duration of excitation of a drug-delivery radiator used to deliver the particles, in accordance with an embodiment of the invention, varied from 1 to about 5 minutes. For a sample of the rats for which relatively homogeneous distribution of IO particles was observed, MRI imaging was used to track concentration of IO particles in their brains over a period of up to 6 weeks. During an initial period of about 4 days following treatment, IO particle concentration in the rats' brains appeared to decreases by estimated amounts of between about 20% and about 30%. Thereafter, for the remainder of the 6 week study, the IO particle concentration remained relatively stable. No cyctotoxic effects in the rats' brain tissue were detected in the MRI images used to track IO concentration. The study indicates that using apparatus and methods in accordance with an embodiment of the invention it is possible to implant drug carrying particles in tissue for release of the drug to the tissue over relatively extended periods of time.

Dimension of drug-delivery radiators can be different from those noted in the above description and can be tailored as needed to the dimensions and constraints of a site to which a drug is to be delivered and/or a region through which the radiator is transported to the site.

In accordance with some embodiments of the invention, a drug-delivery radiator is “transported” to a site to which it is desired to deliver a drug in a compressed state so that during transport it occupies a volume that is characterized by at least one relatively small dimension convenient for transport. At the site, the drug-delivery radiator is expanded to better conform to dimensions of the site.

For example, if it is desired to deliver a drug to the walls of the bladder, which has a relatively large volume, it can be advantageous to thread a drug-delivery radiator through the urethra in a compressed state and once inside the bladder to expand the radiator to a size that provides for better dispersion of the drug to the walls of the bladder. Or, if a drug-delivery radiator is to be threaded through the vascular system to deliver a drug to a relatively large blood vessel it can be advantageous to thread the radiator through the system in a compressed form and expand the radiator at the site of the large blood vessel to deliver the drug.

In some embodiments of the invention, an expandable drug-delivery radiator is a spring radiator coupled to a catheter wire that is transported inside a catheter to a site at which it is to be used to disperse a drug. During transportation inside the catheter, the coils of the spring radiator are compressed so that they have a relatively small diameter and at the site, the radiator is pushed out the distal end of the catheter enabling the coils to expand to a diameter larger than their diameter in the compressed state. Optionally, the catheter wire is constructed using methods known in the art so that it has sufficient “pushability” to push the spring out of the catheter. In some embodiments of the invention, the catheter comprises an additional “push rod”, which is used to aid in pushing the spring radiator out of the catheter. After the drug-delivery radiator disperses a drug to the site, it is drawn back into the catheter and removed from the site with the catheter.

FIGS. 6A-6D schematically show an expandable drug-delivery spring radiator 200 having coils 202 being used to deliver a drug to a region 204 of a large or enlarged blood vessel 206 having walls 208, after being threaded through the vascular system within a catheter 210, in accordance with an embodiment of the invention. Spring radiator 200 is optionally formed as an integral part of a catheter wire 211. Large or enlarged blood vessel 206 may for example be an aorta or an aneurism in a blood vessel.

FIG. 6A schematically shows drug-delivery radiator 200 with its coils 202 compressed within a distal end 212 of catheter 210, which is being threaded through blood vessel 206 to region 204. In FIG. 6B radiator 200 has been pushed out distal end 212 of catheter 210, as a result of which, coils 202 expand to a diameter larger than the internal diameter of the lumen of catheter 210 to which they were constrained inside the catheter. In the figure, expanded spring radiator 200 is shown excited to vibrate and disperse a drug 220 in directions indicated by arrows 222 to walls 208 of blood vessel 206 in region 204.

In some embodiments of the invention, a layer of particles comprising the drug is adhered to surfaces of spring radiator 200 using methods similar to those described above. Optionally, the internal surface of catheter 210 and or surfaces of spring radiator 200 are shaped to reduce possible damage to the drug layer when the radiator is pushed out of the catheter. For example, the inside surface of catheter 210 may be formed with a small number of longitudinal ridges that so that spring radiator 200 contacts only a relative small surface area inside the catheter. Additionally or alternatively, the wire in spring radiator 200 may be optionally shaped so that the outer surfaces of coils 202 that face and contact the inside surface of catheter 210 are concave. Drug particles adhered to spring radiator 200 “nestle” in the valleys of the concave surfaces and are protected from being damaged by contact with the inside surface of catheter 210. Similarly coils 202 may be shaped with protruding nubs that contact the inside surface of catheter 210 and protect most of the surface of the coils from contact with inside surface of the catheter.

FIG. 6C schematically shows an enlarged cross-sectional view of catheter 210 formed with internal ridges 214 that contact spring radiator 200 at only a small number of locations along a coil 202 of the spring and prevent a layer, indicated by dashed lines 224, of drug 220 from being damaged by contact with the catheter. By way of example, in FIG. 6C four ridges 214 are shown. A number of ridges different from four, for example three, may be used in the practice of the invention.

In some embodiments of the invention a liquid, such as a suitable saline solution comprising drug particles 220 is flushed through catheter 210 to spring radiator 200 while it is vibrating to deliver the drug particles by sonophoresis to walls 208. For situations in which spring radiator 200 is used in a bodily fluid, such as blood or urine, drug particles may be delivered, using methods known in the art, to the neighborhood of radiator 200 for dispersion by sonophoresis without use of carrier liquid. The inventors have found that a spring radiator in accordance with an embodiment of the invention is relatively efficient in dispersing particles comprised in a liquid to a site by sonophoresis.

In some embodiments of the invention, a drug-delivery radiator is positioned in or near to a tissue without having a drug adhered to the radiator and the radiator is excited to vibrate and disperse a drug, which is transported to a neighborhood of the radiator, by sonophoresis to the tissue.

FIG. 6D schematically shows expandable spring radiator 200 being withdrawn into catheter 210 by pulling on catheter wire 211 in preparation for removing the radiator from blood vessel 206. As spring radiator 200 is withdrawn into catheter 210 coils 202 are “stretched out”, as is schematically shown in the figure.

In some embodiments of the invention, a compressible spring radiator is formed having a shape and construction similar to that of a stent. The “stent radiator” is delivered to a site inside a catheter and expanded similarly to the manner in which, for example, a vascular a stent is positioned and expanded at a desired location in a blood vessel. The stent vibrator is coupled to at least one catheter wire that enables the stent to be withdrawn back into the catheter after it has been excited to vibrate and disperse a drug to the site.

Whereas exemplary embodiments of drug-delivery horns are shown being used with an isolation jacket and spring radiators without an isolation jacket, any drug-delivery radiator in accordance with an embodiment of the invention, may be used with or without an isolation jacket as circumstances may indicate. When contact with tissue into which the drug-delivery vibrator is introduced substantially damps vibrations in the radiator, it is generally advantageous to operate the vibrator with an isolation jacket and isolation liquid. When contact with tissue, such as with blood or urine, does not in general substantially damp vibrations in the radiator, the radiator may, optionally, be used without an isolation jacket.

In the above description, a drug-delivery radiator is shown being used to disperse an anticancer drug to a tumorous site of a patient's brain, or to deliver a thrombolyte. However, the invention is not limited to such applications and others noted in the description such as dispersing a drug to the bladder. A drug-delivery radiator in accordance with an embodiment of the invention may be used to disperse drugs other than anticancer drugs, thrombolytes and anti-restenosis drugs and to deliver drugs to sites other than those noted. For example, a spring radiator similar to that shown in FIG. 3A may be used to treat a tumor in a region of the liver or deliver a substance that stimulates angiogenesis to a region of heart tissue. By way of another example methods and apparatus in accordance with an embodiment of the invention may be used to advantage in dispersing a pain killing drug to a desired site. For example, apparatus and methods in accordance with an embodiment of the invention may be used for intrathecal delivery of a pain killing drug.

It is noted that whereas exemplary embodiments of the invention have been described as delivering therapeutic drugs for treating disease or disease states, they are of course not limited to delivering only drugs. They may in general be used to deliver any beneficial substance such as for example vitamins, stimulants, cosmetic agents or disease prevention substances and the word drug as used herein is intended to indicate all such substances.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art. The scope of the invention is limited only by the following claims.

1. Apparatus for delivering a drug to a target site of a body comprising:
a dispersing member adapted to vibrate when acoustically excited;
a source of acoustic energy controllable to couple acoustic energy to the dispersing member to excite it to vibrate; and
a drug adhered to the dispersing member so that when the acoustic source excites the dispersing member, the drug is dispersed therefrom.

2. Apparatus according to claim 2 wherein the dispersing member comprises an elongate body having an axis along its long direction.

3. Apparatus according to claim 2 wherein the dispersing member is characterized by relatively abrupt changes in its cross section perpendicular to the axis as a function of position along the axis.

4. Apparatus according to claim 1 wherein the dispersing member comprises a plurality of relatively large cross section regions separated by relatively small cross section regions.

5. Apparatus according to claim 4 wherein the relatively large cross section regions have chamfered edges.

6. Apparatus according to claim 1 wherein the dispersing member comprises a plurality of cone shaped sections having relatively small first ends and relatively large second ends.

7. Apparatus according to claim 6 wherein the first ends face a same direction.

8. Apparatus according to claim 7 wherein the size of the cone shaped sections decrease as a function of distance along the dispersing member axis in the direction along which the first ends face.

9. Apparatus according to claim 2 wherein the dispersing member has a spiral screw shape.

10. Apparatus according to claim 1 wherein the dispersing member is integrally formed as a portion of a catheter wire.

11. Apparatus according to claim 10 and comprising a catheter that comprises the catheter wire.

12. Apparatus according to claim 1 wherein the dispersing member comprises a spring having at least one coil formed from a wire and an axis.

13. Apparatus according to claim 12 wherein the at least one coil comprises a plurality of coils.

14. Apparatus according to claim 13 wherein all of the coils have a same size.

15. Apparatus according to claim 13 wherein adjacent coils have different size.

16. Apparatus according to claim 13 wherein non-adjacent coils have a same size.

17. Apparatus according to 13, wherein the coils comprise at least one relatively large first coil and at least one relatively large second coil and at least one intermediate coil smaller than the at least one first and at least one second coil located between them.

18. Apparatus according to claim 17 and comprising a barrier adhered between the at least one first coil and the at least one second coil.

19. Apparatus according to claim 18 wherein the barrier forms a surface having a lumen in which the at least one intermediate coil is located.

20. Apparatus according to claim 13 wherein the spring has a tapered shape in which the size of its coils decrease along a direction from a first end of the spring to a second end of the spring.

21. Apparatus according to claim 13 wherein the coils have a constant pitch.

22. Apparatus according to claim 13 wherein all coils have a same shape.

23. Apparatus according to claim 12 wherein a coil of the at least one coil is circular.

24. Apparatus according to claim 12 wherein the dispersing member is integrally formed as a portion of a catheter wire.

25. Apparatus according claim 24 and comprising a catheter that comprises the catheter wire.

26. Apparatus according to claim 12 wherein the coils of the spring are expandable.

27. Apparatus according to claim 26 and comprising a housing in which the spring may be housed with its coils compressed and from which it may be removed enabling the coils to expand.

28. Apparatus according to claim 27 wherein the housing comprises ridges that are substantially parallel to the axis of the spring dispersing member and contact at least some of the compressed coils when the dispersing member is housed in the housing.

29. Apparatus according to claim 27 and comprising a catheter wherein the housing comprises a portion of the catheter

30. Apparatus according to claim 12 wherein the dispersing member is integrally formed as a portion of a catheter wire.

31. Apparatus according to claim 1 wherein the dispersing member has a stent-like configuration.

32. Apparatus according claim 31 wherein the stent-like configuration has a compressed and an expanded state.

33. Apparatus according claim 32 and comprising a housing in which the dispersing member may be housed in its compressed state and from which it may be removed and changed into its expanded state.

34. Apparatus according to claim 1 and comprising a jacket in which the dispersing member is positioned that has at least one exit port formed therein through which particles of the substance dispersed by the dispersing member exit.

35. Apparatus according to claim 34 wherein the jacket is filled with a liquid, which when the dispersing member is positioned in the site or a neighborhood therof, protects the dispersing member from contact with material at the site or in the neighborhood.

36. Apparatus according to claim 1 wherein the source of acoustic energy couples at least one pulse of acoustic energy to the dispersing member and controls at least one characteristic of the at least one acoustic pulse to control dispersion of the substance.

37. Apparatus according to claim 36 wherein the acoustic source controls the at least one characteristic to control kinetic energy of particles of the substance dispersed from the dispersing member.

38. Apparatus according to claim 36 wherein the at least one characteristic comprises amplitude of the at least one acoustic pulse.

39. Apparatus according to claim 37 wherein the at least one characteristic comprises frequency of the at least one acoustic pulse.

40. Apparatus for delivering a drug in a neighborhood of a target site of a body to the site comprising:
a dispersing member comprising at least one coil formed from a wire; and
a source of acoustic energy controllable to couple acoustic energy to the dispersing member to excite it to vibrate; wherein
when the drug and the dispersing member are located in a neighborhood of the site and the acoustic source excites the dispersing member, the dispersing member transmits acoustic waves that tend to propel the substance to the site.

41. Apparatus for delivering a drug in a neighborhood of a target site of a body to the site comprising:
a dispersing member comprising an elongate screw shaped body; and
a source of acoustic energy controllable to couple acoustic energy to the dispersing member to excite it to vibrate; wherein
when the drug and the dispersing member are located in a neighborhood of the site and the acoustic source excites the dispersing member, the dispersing member transmits acoustic waves that tend to propel the substance to the site.

42. Apparatus for delivering a drug in a neighborhood of a target site of a body to the site comprising:
a dispersing member comprising an elongate body having an axis and a cross section perpendicular to the axis that changes relatively abruptly as a function of position along the axis; and
a source of acoustic energy controllable to couple acoustic energy to the dispersing member to excite it to vibrate; wherein
when the drug and the dispersing member are located in a neighborhood of the site and the acoustic source excites the dispersing member, the dispersing member transmits acoustic waves that tend to propel the substance to the site.

43. Apparatus for delivering a drug in a neighborhood of a target site of a body to the site comprising:
an expandable dispersing member having a compressed and an expanded state;
a source of acoustic energy controllable to couple acoustic energy to the dispersing member to excite it to vibrate; wherein
wherein the drug is located in a neighborhood of the site and the dispersing member is transported to the neighborhood in the compressed state and at the neighborhood is transformed to its expanded state and when the acoustic source excites the dispersing member in the expanded state the dispersing member transmits acoustic waves that tend to propel the drug to the site.

44. A method of delivering a drug to a target site of a body comprising:
providing a dispersing member adapted to vibrate when acoustically excited;
adhering a drug to the dispersing member so that when the excited to vibrate the drug is dispersed therefrom;
positioning the dispersing member at the site or a neighborhood thereof, and
acoustically exciting the dispersing member.

45. A method according to claim 44 wherein providing a dispersing member comprises providing an expandable dispersing member having a compressed and an expanded state;

46. A method according to claim 45 wherein positioning the dispersing member comprises transporting the dispersing member to the site or a neighborhood thereof in the compressed state;

47. A method according to claim 46 wherein acoustically exciting the dispersing member comprises transforming the dispersing member to its expanded state at the site or the neighborhood thereof.
